(19) United States
(12) Patent Application Publication
(10) Pub. No.: US 2025/0214927 A1
(43) Pub. Date: Jul. 3, 2025

(54) AN IMPROVED PROCESS FOR PURIFICATION OF ROBENACOXIB

(71) Applicant: ALIVIRA ANIMAL HEALTH LIMITED, Thane (West), Mumbai, Maharashtra (IN)

(72) Inventors: Sharat Pandurang NARASAPUR, Mumbai, Maharashtra (IN); Pejakala Kakrannaya VASUDEVA, Visakhapatnam, Andhra Pradesh (IN); Suresh RAMASAMY, Visakhapatnam, Andhra Pradesh (IN)

(21) Appl. No.: 18/849,943

(22) PCT Filed: Mar. 9, 2023
(86) PCT No.: PCT/IN2023/050223

§ 371 Date: Sep. 23, 2024

(30) Foreign Application Priority Data
Mar. 23, 2022 (IN) 202221016167

(51) Int. Cl. C07C 227/42

(52) U.S. Cl. CPC C07C 227/42

(57) ABSTRACT

The present invention relates to an improved process for purification of Robenacoxib. More particularly, the present invention relates to a process for obtaining crystalline Robenacoxib in high yield with high purity. Furthermore, the present invention provides an improved process for the purification of Robenacoxib which is essentially free of lactam impurities (less than 0.1%).

### FIELD OF INVENTION

The present invention relates to an improved process for purification of Robenacoxib. More particularly, the present invention relates to a process for obtaining crystalline Robenacoxib in high yield with high purity.

### BACKGROUND & PRIOR ARTS

Robenacoxib (I) is a non-steroidal anti-inflammatory drug (NSAID) of the coxib class which selectively inhibits the cyclooxygenase 2 enzyme (COX-2). is structural analogue to diclofenac. Robenacoxib is a free acid chemically known as {5-Ethyl-2-[(2,3,5,6-tetrafluorophenyl)amino]phenyl}acetic acid OR 2-[5-ethyl-2-(2,3,5,6-tetrafluoroanilino)phenyl]acetic acid and is structurally represented as

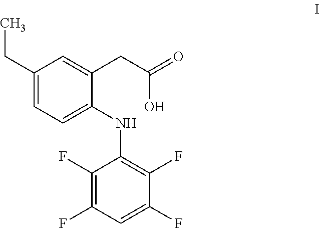 [SMILES: CCc1ccc(Nc2c(F)c(F)cc(F)c2F)c(CC(=O)O)c1]

Robenacoxib is a non-steroidal anti-inflammatory drug (NSAID) used for the treatment of pain and inflammation in pet animals. The drug exhibits properties such as analgesic and anti-inflammatory effects by limiting the synthesis of prostaglandins. Robenacoxib helps to inhibit the cyclooxygenase enzyme, COX-2 which is responsible for synthesizing prostaglandins which causes pain, inflammation, and fever. Robenacoxib selectively inhibits COX-2 enzyme.

Robenacoxib, is marketed as Onsior® tablets in five different strengths (6 mg for cats and 5 mg, 10 mg, 20 mg, and 40 mg for dogs) and as a solution for injection (20 mg/ml for dogs and cats).

Robenacoxib was first disclosed in U.S. Pat. No. 6,291,523 of Elanco Tiergesundheit AG. Example 3 describes the process for the preparation of Robenacoxib and its further crystallization in hexane.

CN109503399 describes another method of preparation of Robenacoxib by secondary Friedel-crafts alkylation method. However, Robenacoxib obtained in the process of CN109503399 is not purified.

It is known that Robenacoxib is practically insoluble in aqueous solution at neutral pH and is freely soluble in aqueous solution in alkaline condition having pH value >8. At acidic pH values, robenacoxib hydrolyses to form a lactam impurity, such as impurity (II).

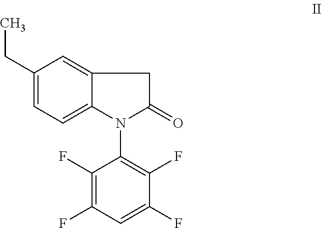 [SMILES: CCc1ccc2c(c1)CC(=O)N2c1c(F)c(F)cc(F)c1F]

Various research groups have attempted the preparation of Robenacoxib by different routes. However, no attempts were made in the past to further purify the Robenacoxib. One such attempt was recently made in EP3830072 of KRKA which describes the process for the preparation of crystallographically pure polymorphic form of Robenacoxib. The solvent system used in EP3830072 is acetone and water, wherein water is used as an anti-solvent. The Robenacoxib obtained from acetone-water purification process, as disclosed in EP3830072, contains more than 0.5% lactam impurity (II) and hence does not comply with VICH guidelines (Veterinary International Co-operation on Harmonization). This is mainly due to acidic nature of crude Robenacoxib. After dissolving crude Robenacoxib in acetone, the pH of the solution is in the range of 2.5-3.5. At this pH, Robenacoxib tends to degrade and form Lactam impurity (II). This degradation results in low yield and less chemical purity of the product, making the process commercially non-viable.

Therefore, there exists a need to develop a process which will eliminate above mentioned drawbacks.

### OBJECTS OF THE INVENTION

Therefore, it is a main object the current invention to provide a process for purification of Robenacoxib.

The further object of the invention is to provide a process for purification of Robenacoxib which reduces degradation of Robenacoxib to lactam impurity.

### SUMMARY OF INVENTION

Accordingly, in an aspect, the present invention provides an improved process for the purification of Robenacoxib.

In another aspect, the present invention provides an improved process for the purification of Robenacoxib which is essentially free of lactam impurities (less than 0.1%).

Crystalline Robenacoxib can be prepared from crude robenacoxib wherein the crude robenacoxib is dissolved in a suitable organic solvent to obtain a clear solution, followed by addition of a precipitating solvent to achieve crystallization of the compound having desired purity and minimum amount of impurity.

These and other features and advantages of the present invention will be apparent from the following detailed description and illustrative embodiments thereof.

### BRIEF DESCRIPTION OF DRAWINGS

FIG. 1 illustrates the XRD pattern/graph of crystalline Robenacoxib obtained by employing the purification process of the present invention.

FIG. 2 illustrates the XRD pattern/graph of the pharmaceutical formulation containing innovator product, that is, product as claimed in U.S. Pat. No. 6,291,523.

FIG. 3 illustrates the XRD pattern of the product as covered in EP3830072.

FIG. 4 illustrates the XRD pattern for a mixture if Form D1 and D2 as covered in EP3830072.

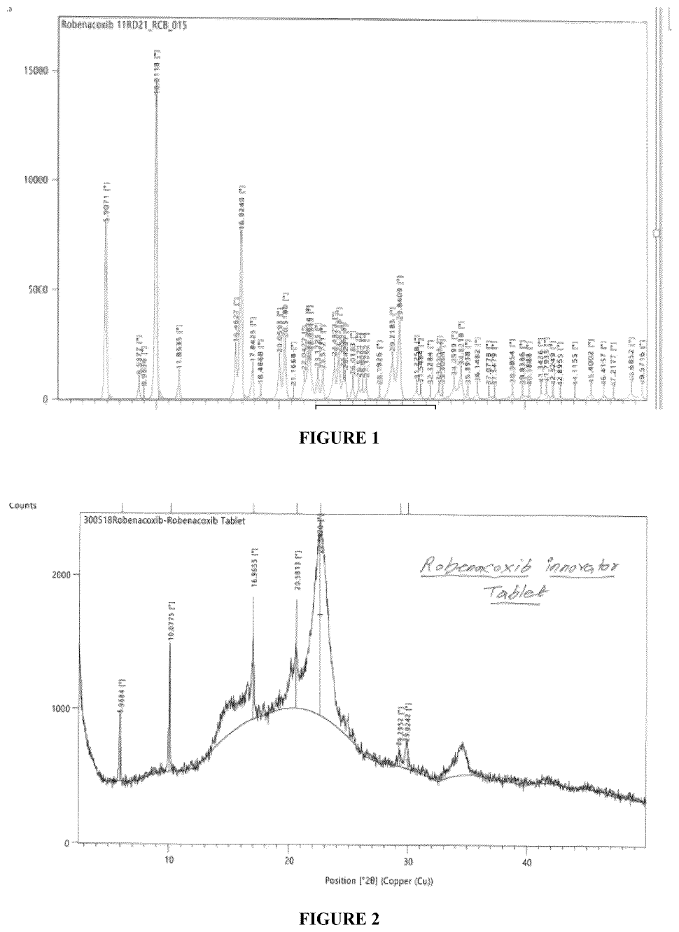

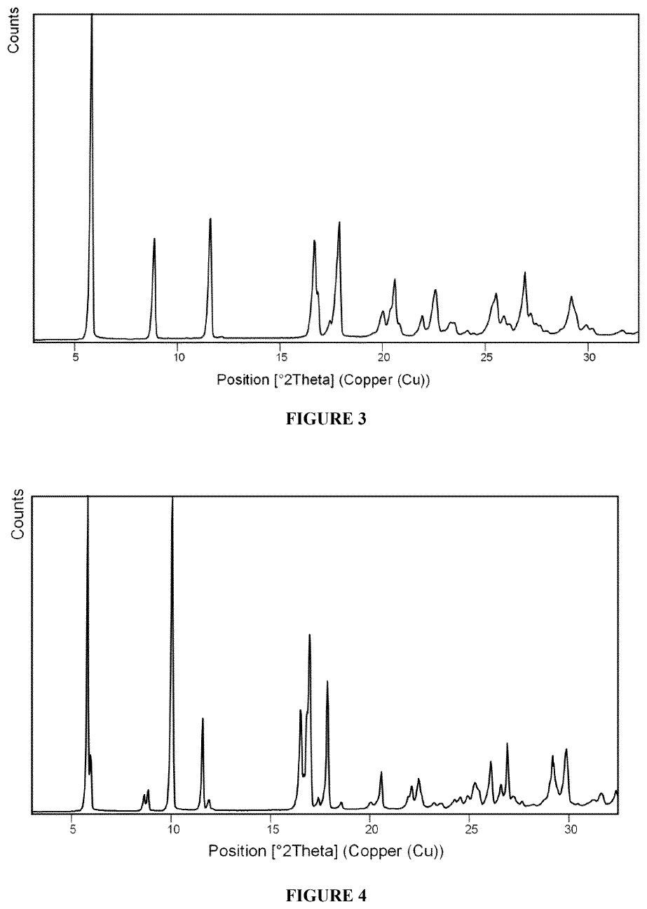

### DETAILED DESCRIPTION OF INVENTION

In order to illustrate the process of the present invention, detailed description of the invention with reference to specific embodiments as described above, is provided along with the illustrative experimental examples. It may be understood for the person skilled in the art that these examples are only typical embodiments of the invention and are not therefore to be considered to be limiting the scope of the present invention.

It will nevertheless be understood that no limitation of the scope of the invention is thereby intended, such alterations and further modifications in the illustrated system, and such further applications of the principles of the invention as illustrated therein being contemplated as would normally occur to one skilled in the art to which the invention relates.

It will be understood by those skilled in the art that the foregoing general description and the following detailed description are exemplary and explanatory of the invention and are not intended to be restrictive thereof.

Reference throughout this specification to “an aspect”, “another aspect” or similar language means that a particular feature, structure, or characteristic described in connection with the embodiment is included in at least one embodiment of the present invention. Thus, appearances of the phrase “in an embodiment”, “in another embodiment” and similar language throughout this specification may, but do not necessarily, all refer to the same embodiment.

The terms “comprises”, “comprising”, or any other variations thereof, are intended to cover a non-exclusive inclusion, such that a process or method that comprises a list of steps does not include only those steps but may include other steps not expressly listed or inherent to such process or method. Similarly, one or more devices or sub-systems or elements or structures or components proceeded by “comprises . . . a” does not, without more constraints, preclude the existence of other devices or other sub-systems or other elements or other structures or other components or additional devices or additional sub-systems or additional elements or additional structures or additional components.

In order to illustrate the process of the present invention, detailed description of the invention with reference to specific embodiments as described above, is provided along with the illustrative experimental examples. It may be understood for the person skilled in the art that these examples are only typical embodiments of the invention and are not therefore to be considered to be limiting the scope of the present invention.

It will nevertheless be understood that no limitation of the scope of the invention is thereby intended, such alterations and further modifications in the illustrated system, and such further applications of the principles of the invention as illustrated therein being contemplated as would normally occur to one skilled in the art to which the invention relates.

It will be understood by those skilled in the art that the foregoing general description and the following detailed description are exemplary and explanatory of the invention and are not intended to be restrictive thereof.

Reference throughout this specification to “an aspect”, “another aspect” or similar language means that a particular feature, structure, or characteristic described in connection with the embodiment is included in at least one embodiment of the present invention. Thus, appearances of the phrase “in an embodiment”, “in another embodiment” and similar language throughout this specification may, but do not necessarily, all refer to the same embodiment.

The terms “comprises”, “comprising”, or any other variations thereof, are intended to cover a non-exclusive inclusion, such that a process or method that comprises a list of steps does not include only those steps but may include other steps not expressly listed or inherent to such process or method. Similarly, one or more devices or sub-systems or elements or structures or components proceeded by “comprises . . . a” does not, without more constraints, preclude the existence of other devices or other sub-systems or other elements or other structures or other components or additional devices or additional sub-systems or additional elements or additional structures or additional components.

The present invention describes an improved process for the purification of Robenacoxib which overcomes the drawbacks in the prior art.

Accordingly, certain embodiments of the present invention relate to a process for purification of crystalline form of Robenacoxib by using a suitable solvent for mixing to form a solution; adding a precipitating solvent to the solution to crystallise the product slowly at lower temperatures; and isolating the crystalline form having minimum or negligible amount of lactam impurity.

Accordingly, in an embodiment, the process for purification of Robenacoxib comprises the steps of:
a) charging Robenacoxib in a suitable container;
b) dissolving Robenacoxib of step (a) in a first solvent, at temperature ranging from 15-25° C., under stirring;
c) checking the pH of the solution of step (b), if pH is below 4.0, then the pH of the reaction mass is adjusted to pH 4.0-5.0, using a suitable pH adjusting reagent at 15-25° C.;
d) stirring the reaction mass of step (c) at 15-25° C. for 20 to 30 minutes till the solution stabilizes at pH 4.0-5.0;
e) filtering the reaction mass of step (d) through hyflo bed at 15-25° C.;
f) washing the reactions mass of step (e) with the first solvent;
g) heating the reaction mass to 50-55° C.
h) charging precipitating solvent at 50-55° C. to the solution of step (g) and cool it further to 0-5° C.;
i) Stirring the above solution for 2 hrs at 0-5° C.;
j) Filtering the solids from the solution of step (i);
k) Washing the solids of step (j) with 1:1 chilled mixture of 1^{st} solvent; precipitating solvent;
l) charging the wet cake obtained in step (k) with the second solvent and stir for 1 hr. at 0-5° C.;
m) Filtering the solids of step (1);
n) Washing the solids of step (m) with chilled second solvent, and
o) Drying the washed solid of step (n) under vacuum, at 50-60° C. for 6 hrs.

In accordance with the above embodiment, the solvents used in step a) to step f), referred to as “first solvent”, in the present invention are selected from, C₁-C₄ alcohols, ethyl acetate, isopropyl acetate, propyl acetate, and butyl acetate, acetonitrile, alkanones such as acetone, butanone, methyl ethyl ketone and methyl propyl ketone, or mixtures of two or more solvents. More preferred solvents for the purpose of present invention are selected from, acetone, toluene, n-Hexane and mixture of two or more of these solvents. Most preferred solvent for making a solution in the initial steps, step a) to f) is acetone.

In another preferred embodiment, the crystallization in step g) is to be performed by precipitation. The second solvent acts as precipitation solvent used step g) to step j) is selected from water, ethers, C₆-C₈-alkanes, C₆-C₈-cycloalkanes including aromatic solvents such as toluene and xylene and mixtures thereof. Preferably, the precipitating solvent used in the present invention process is water.

For the purposes of present invention, the third solvent, referred to as “aromatic hydrocarbon”, employed in the process steps l) to n) is selected from aromatic hydrocarbons such as benzene, toluene and xylene, with toluene being particularly preferred solvent.

According to the observation of the inventors of the present invention, the pH of the solution in the initial steps forms a critical part of the process since Robenacoxib tends to degrade at the acidic pH. The optimum pH conditions of the initial step a) to step f) are between pH value 4.0 to 5.0 which is maintained by using pH adjusting reagent.

Accordingly, pH adjustment reagent can be selected from but are not limited to, aqueous ammonia, aqueous sodium hydroxide, and the like. Preferably, the pH adjustment reagent used is aqueous ammonia.

Typically, the crystallizing temperature for obtaining the pure form of Robenacoxib ranges from 0° C. to about 10° C., preferably in the range of 0° C. to 5° C. The crystallizing time required for the complete crystallizing of Robenacoxib in step g) and h) is about 1 to 5 hours, preferably about 1 to 2 hours.

In one of the embodiments, for each part by weight of robenacoxib 3 to 5 parts by weight, more preferably 3 parts by weight of first solvent is used in step a).

In another embodiment for each part by weight of robenacoxib 3 to 7 parts by weight, more preferably 5 parts by weight of precipitating solvent is used in step h) for crystallization.

In another embodiment for each part by weight of robenacoxib 0.5 to 3 parts by weight, more preferably 1 part by weight of second solvent is used in step l) for crystallization.

The process, in accordance with the above embodiments, leads to formation of a mixture of Form D1 and D2.

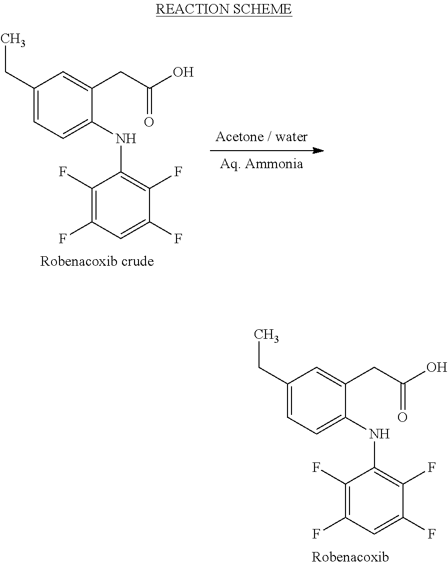 [SMILES: CCc1ccc(Nc2c(F)c(F)cc(F)c2F)c(CC(=O)O)c1 ; CCc1ccc(Nc2c(F)c(F)cc(F)c2F)c(CC(=O)O)c1]

### EXAMPLES

The present invention is demonstrated by way of following examples. It is to be understood for a person skilled in the art however, that they are not to be construed as limiting the scope of the invention in any way.

### Example 1: Without Using Aq. Ammonia

i. Dissolve Robenacoxib crude (20 g) in 80 mL acetone at 25-30° C.,
ii. Add 1 g of activated carbon at 25-30° C.,
iii. Filter the reaction mass through hyflo bed and wash the hyflo bed with 20 mL acetone,
iv. Charge Filtrate in Round bottom flask and heat the reaction mass to 50-55° C.,
v. Add 100 mL water slowly by using a dropping funnel at 50-55° C.,
vi. Cool the reaction mass to 0-5° C. and filter the solid and wash with 1:1 acetone-water,
vii. Dry the wet material at 40-50° C. for 4 hrs to afford 16.0 g of pure Rbenacexib-Robenacoxib (Yield: 80.0%).

### Example 2: With Using Aq. Ammonia

i. Dissolve Robenacoxib crude (20 g) in 80 mL acetone at 25-30° C.,
ii. Adjust the pH of the reaction mass to 4.0-5.0 using aqueous ammonia (3 mL),
iii. Add 1 g of activated carbon at 25-30° C.,
iv. Filter the reaction mass through hyflo bed and wash the hyflo bed with 20 mL acetone,
v. Charge Filtrate in round bottom flask and heat the reaction mass to 50-55° C.,
vi. Add 100 mL water slowly by using dropping funnel at 50-55° C.,
vii. Cool the reaction mass to 0-5° C. and filter the solid with 1:1 acetone-water,
viii. Charge the wet material obtained in step vii) in a round bottom flask,
ix. Charge toluene (20 mL) and stir for 1 hrs at 0-5° C.,
x. Filter the solid and wash with toluene (10 mL),
xi. Dry the wet material at 40-50° C. for 4 hrs to afford 17 g of pure Rebenacoxib (Yield: 85.0%).

RESULTS OF EXAMPLE 1 & 2

| Sr. | | HPLC | Lactam Impurity | |
|---|---|---|---|---|
| No. | Example details. | Purity (%) | (%) | Yield (%) |
| 1 | Example-1 (without | 99.18 | 0.73 | 80 |
| | using aq. Ammonia) | | | |
| 2 | Example- 2 (using aq. | 99.77 | 0.07 | 85 |
| | Ammonia) | | | |

### MAIN ADVANTAGES OF THE PRESENT INVENTION

Robenacoxib is obtained in higher yield and high chemical purity (99.80%).
Lactam impurity (II) formation is significantly less (NMT 0.10%)
Process is simpler, economical and commercially viable.
Due to higher purity of the product, the process of the present invention avoids the reprocess in commercial batches.

We claim:

1. A process for purification of Robenacoxib crystalline form in high yield and high chemical purity, wherein the process comprises:
a. dissolving Robenacoxib in a first solvent, at temperature ranging from 15-25° C., under stirring;
b. checking and adjusting the pH of the solution of step (a) to pH 4.0-5.0, by using a suitable pH adjusting reagent, at 15-25° C.;
c. filtering the reaction mass of step (b) through hyflo bed at 15-25° C.;
d. heating the reaction mass of Step (c) to 50-55° C.;
e. charging a second solvent at 50-55° C. to the solution of step (d) and cooling it further to 0-10° C.;
f. filtering and washing the solids of step (e) with 1:1 chilled mixture of first solvent: second solvent;
g. charging the wet cake obtained in step (f) with an aromatic hydrocarbon and stirring for 1 hr. at 0-5° C.;
h. filtering the solids of step (g);
i. washing the solids of step (h) with chilled third solvent, and
j. drying the washed solid of step (i) under vacuum, at 50-60° C. for 6 hrs,

2. The process as claimed in claim 1, wherein the suitable pH adjusting agent is selected from but are not limited to, aqueous ammonia, aqueous sodium hydroxide, and the like.

3. The process as claimed in claim 2, wherein the suitable pH adjusting agent is aqueous ammonia.

4. The process as claimed in claim 1, wherein the amount of lactam impurity produced is in range of 0.05-0.1%.

5. The process as claimed in claim 1, wherein, the first solvent is selected from group consisting of C₁-C₄ alcohols, ethyl acetate, isopropyl acetate, propyl acetate, and butyl acetate, acetonitrile, alkanones such as acetone, butanone, methyl ethyl ketone and methyl propyl ketone, or mixtures of two or more solvents.

6. The process as claimed in claim 5, wherein the first solvent is preferably selected from acetone, toluene and mixture of two or more of these solvents.

7. The process as claimed in claim 6, wherein the first solvent is acetone.

8. The process as claimed in claim 7, wherein for each part by weight of robenacoxib 3 to 5 parts by weight first solvent is used in step a).

9. The process as claimed in claim 8, for each part by weight of robenacoxib 3 parts by weight of first solvent is used in step a).

10. The process as claimed in claim 1, wherein, the second solvent is selected from group consisting of water, ethers, C₆-C₈-alkanes, C₆-C₈-cycloalkanes including aromatic solvents such as toluene and xylene and mixtures thereof.

11. The process as claimed in claim 10, wherein the second solvent is water.

12. The process as claimed in claim 11, wherein for each part by weight of robenacoxib 3 to 7 parts by weight of second solvent is used.

13. The process as claimed in claim 12, for each part by weight of robenacoxib 5 parts by weight of the second solvent is used in step e) for crystallisation.

14. The process as claimed in claim 1, wherein the aromatic hydrocarbon is selected from group consisting of benzene, toluene and xylene.

15. The process as claimed in claim 14, wherein the aromatic hydrocarbon is toluene.

16. The process as claimed in claim 15, each part by weight of robenacoxib 0.5 to 3 parts by weight aromatic hydrocarbon is used for crystallisation.

17. The process as claimed in claim 16, each part by weight of robenacoxib preferably 1 part by weight of second solvent is used in step g) for crystallisation.

18. The process as claimed in claim 1, wherein the robenacoxib is obtained as Form D2.
